(19) United States
(12) Patent Application Publication
(10) Pub. No.: US 2007/0191931 A1
(43) Pub. Date: Aug. 16, 2007

(54) Bioerodible endoprostheses and methods of making the same

(76) Inventors: Jan Weber, Maple Grove, MN (US); Liliana Atanasoska, Edina, MN (US); Alexey Kondyurin, Sutherland (AU)

Correspondence Address: FISH & RICHARDSON PC, P.O. BOX 1022, MINNEAPOLIS, MN, 55440-1022, US

(21) Appl. No.: 11/355,368

(22) Filed: Feb. 16, 2006

(51) Int. Cl. A61F 2/06; A61L 33/00

(52) U.S. Cl. 623/1.38; 623/1.46; 427/2.25

(57) ABSTRACT

Endoprostheses include a wall having a base, e.g., a bioerodible base, and a polymer that may include a region of carbonized polymer formed by implantation.

### TECHNICAL FIELD

This disclosure relates to bioerodible endoprostheses, and to methods of making the same.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire disclosure of which is hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

It is sometimes desirable for an implanted endoprosthesis to erode over time within the passageway. For example, a fully erodible endoprosthesis does not remain as a permanent object in the body, which may help the passageway recover to its natural condition.

### SUMMARY

This disclosure relates to bioerodible endoprostheses, and to methods of making the same. The endoprostheses can, e.g., provide surfaces which support cellular growth. Many of the endoprostheses disclosed can be configured to erode in a controlled and predetermined manner in the body and/or can be configured to deliver therapeutic agents in a controlled and predetermined manner to specific locations in the body.

In one aspect, the disclosure features an endoprosthesis that includes an endoprosthesis wall having a bioerodible base and a region including carbonized polymer material.

In another aspect, the disclosure features a method of making an endoprosthesis that includes providing an endoprosthesis that includes a bioerodible base and a polymer, and treating the polymer by ion implantation.

In another aspect, the disclosure features a method of making an endoprosthesis, that includes providing an endoprosthesis having a metal base and having a polymer layer, and treating the polymer layer by ion implantation.

In another aspect, the disclosure features endoprostheses that exhibit a D peak and/or a G peak in Raman.

In another aspect, the disclosure features an endoprosthesis that is filled with one or more therapeutic agents, treated with one or more therapeutic agents, and/or has a fractured surface morphology, as described herein, in which fractures include one or more therapeutic agents.

Other aspects or embodiments may include combinations of the features in the aspects above and/or one or more of the following. The base is a bioerodible polymer system. The carbonized polymer material is an integral modified region of the base bioerodible polymer system. The base is a bioerodible metal. The region includes a diamond-like carbon material. The region includes a graphitic carbon material. The region includes a region of crosslinked base polymer material. The crosslinked region is directly bonded to the carbonized polymer material and to substantially unmodified base polymer material. The endoprosthesis includes a region of oxidized polymer material, the oxidized region being directly bonded to the carbonized material without further bonding to the base. The region extends from a surface of the base. An overall modulus of elasticity of the base is within about +/−10% of the base polymer system without the region. A thickness of the region is about 10 nm to about 2000 nm. The region has a thickness that is about 20% or less than an overall thickness of the base polymer system. The base polymer is selected from the group consisting of polyester amides, polyanhydrides, polyorthoesters, polylactides, polyglycolides, polysiloxanes, cellulose derivatives, and copolymers or blends of any of these polymers. The base is a metal, e.g., magnesium, calcium, lithium, rare earth elements, iron, aluminum, zinc, manganese, cobalt, copper, zirconium, titanium, or mixtures or alloys of any of these metals. The region has a fractured surface morphology having a surface fracture density of about 5 percent or more. The region carries a therapeutic agent. The base includes a coating. The base is a polymer and the base is treated to provide a modified region. The bioerodible base is provided with a polymer layer, and the polymer layer is treated to provide a modified region.

All publications, patent applications, patents, and other references mentioned herein are incorporated by reference herein in their entirety.

Aspects and/or embodiments may have one or more of the following advantages. The endoprostheses may not need to be removed from a lumen after implantation. The endoprostheses can have a low thrombogenecity. Lumens implanted with the endoprostheses can exhibit reduced restenosis. The hard surfaces and/or oxidized surfaces provided by the endoprostheses support cellular growth (endothelialization) and, as a result, minimizes the risk of endoprosthesis fragmentation. The hard surfaces provided are robust, having a reduced tendency to peel from bulk material. The hard surfaces provided are flexible. The rate of release of a therapeutic agent from an endoprosthesis can be controlled. The rate of erosion of different portions of the endoprostheses can be controlled, allowing the endoprostheses to erode in a predetermined manner, reducing, e.g., the likelihood of uncontrolled fragmentation. For example, the predetermined manner of erosion can be from an inside of the endoprosthesis to an outside of the endoprosthesis, or from a first end of the endoprosthesis to a second end of the endoprosthesis.

An erodible or bioerodible endoprosthesis, e.g., a stent, refers to an endoprosthesis, or a portion thereof, that exhibits substantial mass or density reduction or chemical transformation, after it is introduced into a patient, e.g., a human patient. Mass reduction can occur by, e.g., dissolution of the material that forms the endoprosthesis and/or fragmenting of the endoprosthesis. Chemical transformation can include oxidation/reduction, hydrolysis, substitution, and/or addition reactions, or other chemical reactions of the material from which the endoprosthesis, or a portion thereof, is made. The erosion can be the result of a chemical and/or biological interaction of the endoprosthesis with the body environment, e.g., the body itself or body fluids, into which it is implanted and/or erosion can be triggered by applying a triggering influence, such as a chemical reactant or energy to the endoprosthesis, e.g., to increase a reaction rate. For example, an endoprosthesis, or a portion thereof, can be formed from an active metal, e.g., Mg or Ca or an alloy thereof, and which can erode by reaction with water, producing the corresponding metal oxide and hydrogen gas (a redox reaction). For example, an endoprosthesis, or a portion thereof, can be formed from an erodible or bioerodible polymer, or an alloy or blend erodible or bioerodible polymers which can erode by hydrolysis with water. The erosion occurs to a desirable extent in a time frame that can provide a therapeutic benefit. For example, in embodiments, the endoprosthesis exhibits substantial mass reduction after a period of time which a function of the endoprosthesis, such as support of the lumen wall or drug delivery is no longer needed or desirable. In particular embodiments, the endoprosthesis exhibits a mass reduction of about 10 percent or more, e.g. about 50 percent or more, after a period of implantation of one day or more, e.g. about 60 days or more, about 180 days or more, about 600 days or more, or 1000 days or less. In embodiments, the endoprosthesis exhibits fragmentation by erosion processes. The fragmentation occurs as, e.g., some regions of the endoprosthesis erode more rapidly than other regions. The faster eroding regions become weakened by more quickly eroding through the body of the endoprosthesis and fragment from the slower eroding regions. The faster eroding and slower eroding regions may be random or predefined. For example, faster eroding regions may be predefined by treating the regions to enhance chemical reactivity of the regions. Alternatively, regions may be treated to reduce erosion rates, e.g., by using coatings. In embodiments, only portions of the endoprosthesis exhibits erodibilty. For example, an exterior layer or coating may be erodible, while an interior layer or body is non-erodible. In embodiments, the endoprosthesis is formed from an erodible material dispersed within a non-erodible material such that after erosion, the endoprosthesis has increased porosity by erosion of the erodible material.

Erosion rates can be measured with a test endoprosthesis suspended in a stream of Ringer's solution flowing at a rate of 0.2 m/second. During testing, all surfaces of the test endoprosthesis can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

Other aspects, features, and advantages will be apparent from the description and drawings, and from the claims.

### DESCRIPTION OF DRAWINGS

FIGS. 1A-1C are longitudinal cross-sectional views, illustrating delivery of a polymeric bioerodible stent in a collapsed state, expansion of the stent, and the deployment of the stent.

FIG. 2A is a perspective view of an unexpanded polymeric bioerodible stent having a plurality of fenestrations.

FIG. 2B is a transverse cross-sectional view of the bioerodible stent of FIG. 2A, showing a base and a hard polymer region.

FIG. 2C is a perspective view of the stent in FIG. 2A in the process of eroding.

FIG. 3 is a schematic illustration of the compositional makeup of a portion of the stent wall illustrated in FIGS. 2A and 2B.

FIG. 4A is a schematic cross-sectional view of a plasma immersion ion implantation apparatus.

FIG. 4B is a schematic top view of stents in a sample holder (metal grid electrode partially removed from view).

FIG. 4C is a detailed cross-sectional view of the plasma immersion ion implantation apparatus of FIG. 4A.

FIG. 5A is a transverse cross-sectional view of a bioerodible stent that has a coating. FIG. 5B is a transverse cross-sectional view of the stent of FIG. 5A after modification.

FIG. 5C is a series of micro-Raman spectra of an outermost surface of a stent having an SIBS coating, the bottom spectrum being before PIII treatment, the middle spectrum being after PIII treatment, and the uppermost spectrum being a difference of the before and after.

FIG. 6A is a photomicrograph a polymeric material surface prior to modification.

FIG. 6B is a photomicrograph of a polymeric material surface after modification.

FIG. 6C is a schematic top view of a polymeric material surface after modification, showing fissures and “islands” that are defined by the fissures.

FIG. 7 is a perspective view of a bioerodible stent that has three portions, each portion having a different erosion rate.

FIGS. 7A-7C are transverse cross-sectional view of the stent of FIG. 7, taken along lines 7A-7A, 7B-7B and 7C-7C, respectively.

FIG. 8 is a sequence of perspective views illustrating a method of making the stent of FIG. 7.

FIGS. 9-11 are longitudinal cross-sectional views, illustrating erosion of the bioerodible stent depicted in FIG. 7 within a body lumen.

### DETAILED DESCRIPTION

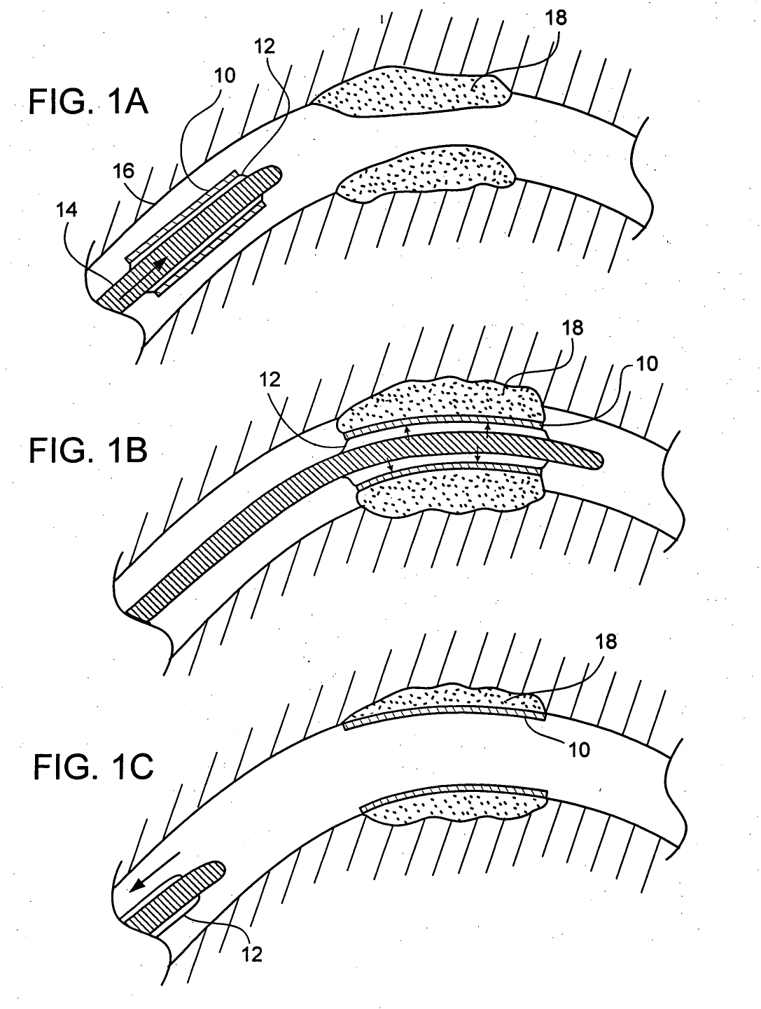

Referring to FIGS. 1A-1C, a bioerodible stent 10 is placed over a balloon 12 carried near a distal end of a catheter 14, and is directed through a lumen 16 (FIG. 1A) until the portion carrying the balloon and stent reaches the region of an occlusion 18. The stent 10 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

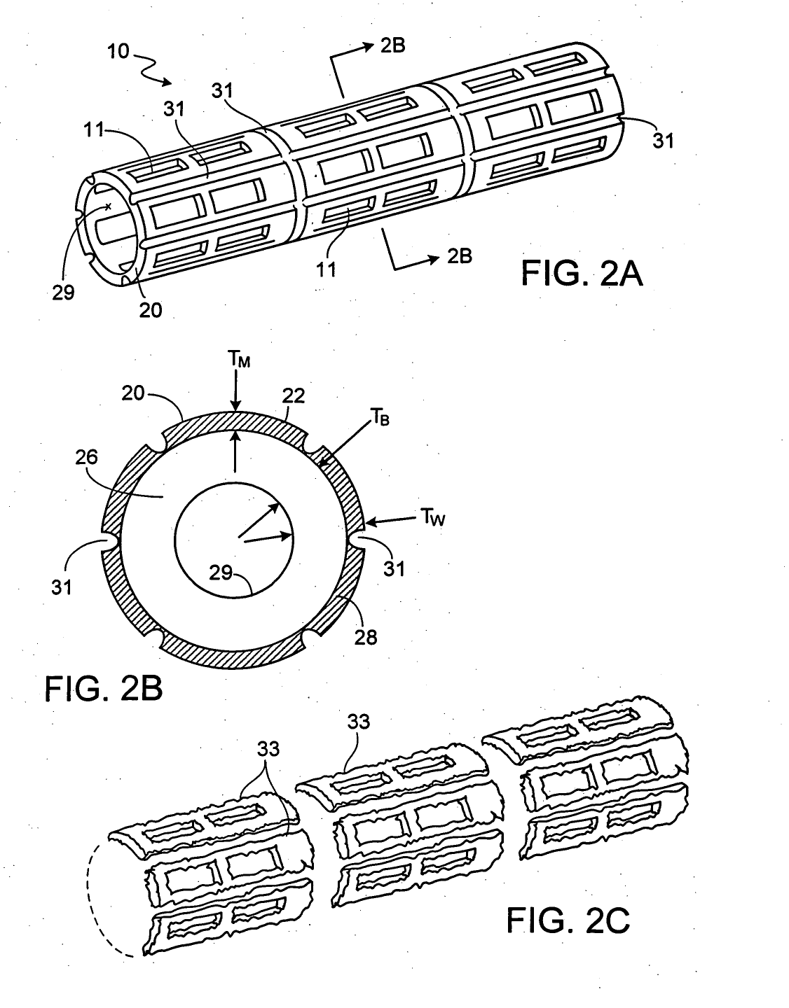

Referring to FIGS. 2A and 2B, bioerodible stent 10 includes a plurality of fenestrations 11 defined in a wall 20. The stent wall 20 is formed of a bioerodible base 26 and a hard polymer region 28 that controls the erosion profile of the base. For example, the hard polymer region 28 is provided over portions of the base in a pattern to shield the base from direct contact with body tissue on a lumen wall, while leaving other portions 31 exposed. Referring as well now to FIG. 2C, the exposed portions 31 degrade more rapidly, resulting in a desired pattern of degradation fragments 33 having a controlled size. The hard polymer region 28 can, e.g., enhance cell growth on outer surfaces of the stent. Region 28 can also enable control over the rate and manner of erosion. For example, the hard polymer region 28 presents a barrier to erosion from the outside, forcing more rapid erosion to occur from the inside 29 of the stent towards the outside of the stent. These advantages can be provided without substantially affecting the overall performance of the stent or the mechanical properties of the base. The base can be formed of a bioerodible base polymer system or a bioerodible metal base system. In the case of a bioerodible base polymer, the hard polymer region can be formed by modifying the base polymer. In the case of a bioerodible metal base, the hard polymer region can be provided on the metal base.

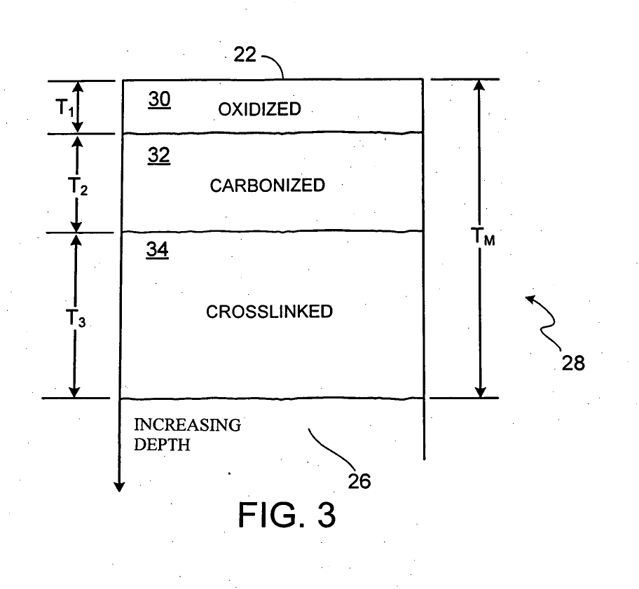

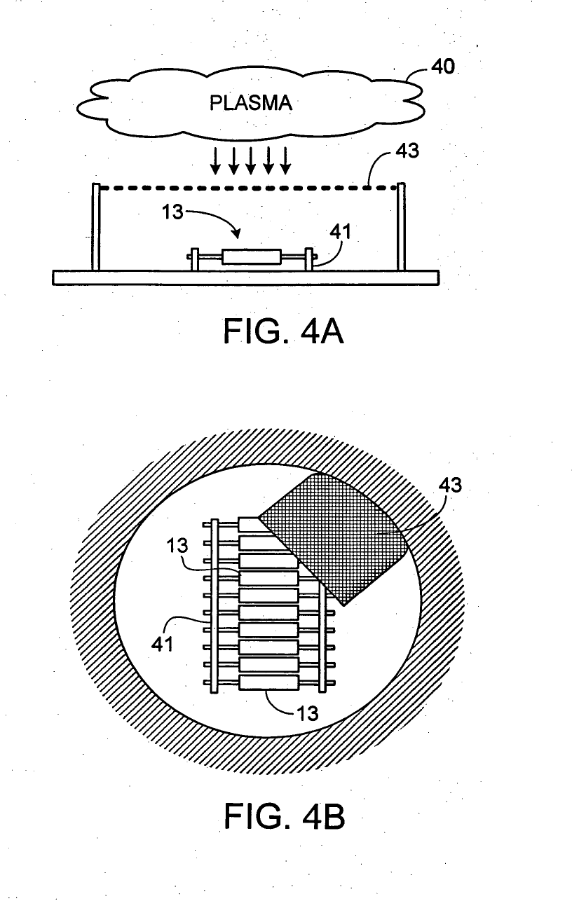

Referring to FIG. 3, the hard polymer region 28 can have a series of sub-regions, including an oxidized region 30 (e.g., having carbonyl groups, aldehyde groups, carboxylic acid groups and/or alcohol groups), a carbonized region 32 (e.g., having increased sp² bonding, particularly aromatic carbon-carbon bonds and/or sp³ diamond-like carbon-carbon bonds), and a crosslinked region 34. The crosslinked region 34 is a region of increased polymer crosslinking that is bonded directly on base polymer system and to the carbonized region 32. The carbonized region 32 is a band that typically includes a high-level of sp³-hybridized carbon atoms, e.g., greater than 25 percent sp³, greater than 40 percent, or even greater than 50 percent sp³−hyridized carbon atoms, such as exists in diamond-like carbon (DLC). The oxidized region 30 that is bonded to the carbonized layer 32 and exposed to atmosphere includes an enhanced oxygen content, relative to the base polymer system. The enhanced oxygen content of the oxidized region offers enhanced hydrophilicity, which can, e.g., enable enhanced cellular overgrowth. The hard nature of the carbonized region can, e.g., enhance cell growth on outer surfaces of the stent and/or can enable control over the rate and manner of bioerosion. The presence of oxidized regions, carbonized regions and crosslinked regions can be detected using, e.g., infrared, Raman and UV-vis spectroscopy. In embodiments, the modified region exhibits D and G peaks in Raman spectra. For example, Raman spectroscopy measurements are sensitive to changes in translational symmetry and are often useful in the study of disorder and crystallite formation in carbon films. In Raman studies, graphite can exhibit a characteristic peak at 1580 cm⁻¹ (labeled ‘G’ for graphite). Disordered graphite has a second peak at 1350 cm⁻¹ (labeled ‘D’ for disorder), which has been reported to be associated with the degree of sp³ bonding present in the material. The appearance of the D-peak in disordered graphite can indicate the presence in structure of six-fold rings and clusters, thus indicating the presence of sp³ bonding in the material. XPS is another technique that has been used to distinguish the diamond phase from the graphite and amorphous carbon components. By deconvoluting the spectra, inferences can be made as to the type of bonding present within the material. This approach has been applied to determine the sp³/sp² ratios in DLC material (see, e.g., Rao, Surface & Coatings Technology 197, 154-160, 2005, the entire disclosure of which is hereby-incorporated by reference herein). Raman spectra of diamond-like carbon materials are also described by Shiao, Thin Solid Films, v. 283, 145-150 (1996), the entire disclosure of which is hereby incorporated by reference herein.

Additional details on detection of hard regions and a suitable balloon for delivery of a stent as described herein, can be found in “MEDICAL BALLOONS AND METHODS OF MAKING THE SAME”, filed concurrently herewith and assigned U.S. patent application Ser. No. ______ [Attorney Docket No. 10527-707001], the entire disclosure of which is hereby incorporated by reference herein.

The graduated, multi-region structure of the hard polymer layer can, e.g., enhance adhesion to the base, reducing the likelihood of delamination. In addition, the graduated nature of the structure and low thickness of the hard polymer region relative to the overall wall thickness enables the wall to maintain many of the advantageous overall mechanical properties of the unmodified wall. Generally, the oxidized region 30 and the carbonized region 32 are not bioerodible, but the crosslinked region 34 is bioerodible, albeit at a slower rate relative to the unmodified base polymer system due, at least in part, to its decreased tendency to swell in a biological fluid. This allows for cells to fully envelope the oxidized and carbonized regions towards the end of the bioerosion process, reducing the likelihood of stent fragmentation.

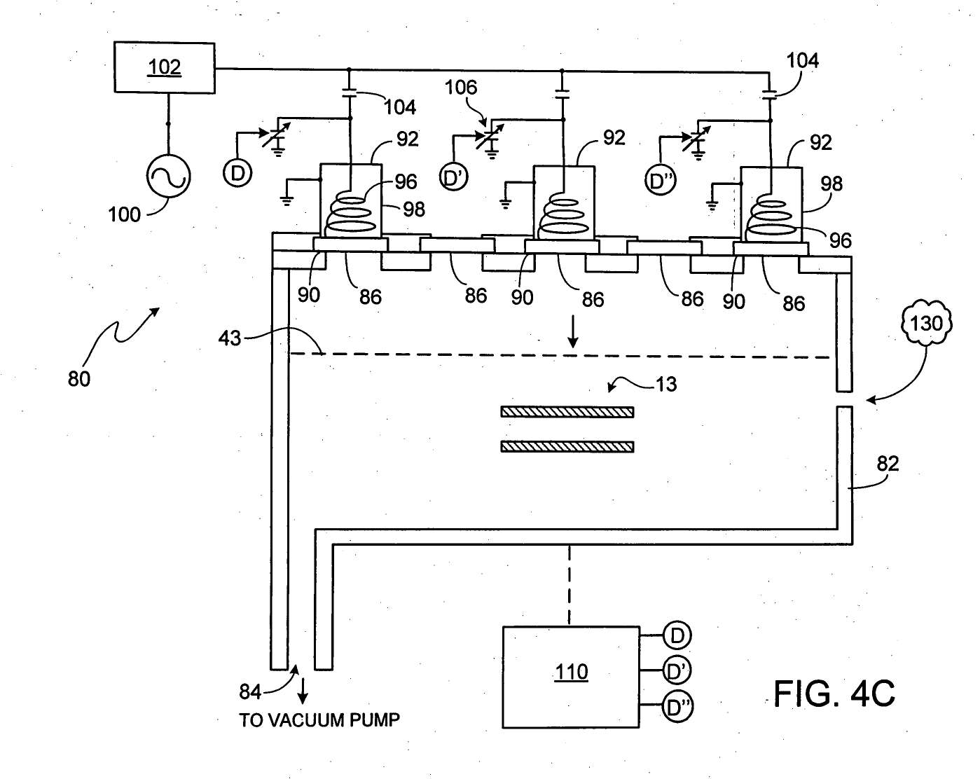

The hard polymer region can be formed, e.g., using an ion implantation process, such as plasma immersion ion implantation (“PIII”). Referring to FIGS. 4A and 4B, during PIII, charged species in a plasma 40, such as a nitrogen plasma, are accelerated at high velocity towards stents 13, which are positioned on a sample holder 41. Acceleration of the charged species of the plasma towards the stents is driven by an electrical potential difference between the plasma and an electrode under the stents. Upon impact with a stent, the charged species, due to their high velocity, penetrate a distance into the stent and react with the material of the stent, forming the regions discussed above. Generally, the penetration depth is controlled, at least in part, by the potential difference between the plasma and the electrode under the stents. If desired, an additional electrode, e.g., in the form of a metal grid 43 positioned above the sample holder, can be utilized. Such a metal grid can be advantageous to prevent direct contact of the stents with the rf-plama between high-voltage pulses and can reduce charging effects of the stent material.

FIG. 4C shows an embodiment of a PIII processing system 80. System 80 includes a vacuum chamber 82 having a vacuum port 84 connected to a vacuum pump and a gas source 130 for delivering a gas, e.g., nitrogen, to chamber 82 to generate a plasma. System 80 includes a series of dielectric windows 86, e.g., made of glass or quartz, sealed by o-rings 90 to maintain a vacuum in chamber 82. Removably attached to some of the windows 86 are RF plasma sources 92, each source having a helical antenna 96 located within a grounded shield 98. The windows without attached RF plasma sources are usable, e.g., as viewing ports into chamber 82. Each antenna 96 electrically communicates with an RF generator 100 through a network 102 and a coupling capacitor 104. Each antenna 96 also electrically communicates with a tuning capacitor 106. Each tuning capacitor 106 is controlled by a signal D, D′, D″ from a controller 110. By adjusting each tuning capacitor 106, the output power from each RF antenna 96 can be adjusted to maintain homogeneity of the generated plasma. The regions of the stent directly exposed to ions from the plasma can be controlled by rotating the stents about their axis. The stents can be rotated continuously during treatment to enhance a homogenous modification of the entire stent. Alternatively, rotation can be intermittent, or selected regions can be masked, e.g., with a polymeric coating, to exclude treatment of those masked regions. Additional details of PIII is described by Chu, U.S. Pat. No. 6,120,260; Brukner, Surface and Coatings Technology, 103-104, 227-230 (1998); Kutsenko, Acta Materialia, 52, 4329-4335 (2004); Guenzel, Surface & Coatings Technology, 136, 47-50, 2001; and Guenzel, J. Vacuum Science & Tech. B, 17(2), 895-899, 1999, the entire disclosure of each of which is hereby incorporated by reference herein.

The type of hard coating region formed is controlled in the PIII process by selection of the type of ion, the ion energy and ion dose. In embodiments, three sub-region are formed, as described above. In other embodiments, there may be more, or less than three sub-regions formed by controlling the PIII process parameters, or by post processing to remove one or more layers by, e.g., solvent dissolution, mechanically removing layers by cutting, abrasion, or heat treating. In particular, a higher ion energy and doses enhances the formation of carbonized regions, particularly regions with hard carbon or DLC or graphite components. In embodiments, the ion energy is about 5 keV or greater, such as 25 keV or greater, e.g. about 30 keV or greater and about 75 keV or less. The ion dosage in embodiments is in the range of about 1×10¹⁴ or greater, such as 1×10¹⁶ ions/cm² or greater, e.g. about 5×10¹⁶ ions/cm² or greater, and about 1×10¹⁸ ions/cm² or less. The oxidized region can be characterized, and the process conditions modified based on FTIR ATR spectroscopy and/or Raman results on carbonyl group and hydroxyl group absorptions. Also, the crosslinked region can be characterized using FTIR ATR spectroscopy, UV-vis spectroscopy and Raman spectroscopy by analyzing C═C group absorptions, and the process conditions modified based on the results. In addition, the process conditions can be modified based on an analysis of gel fraction of the crosslinked region, which can be determined using the principle that a crosslinked polymer is not soluble in any solvent, while a non-crosslinked polymer is soluble in a solvent. For example, the gel fraction of a sample can be determined by drying the sample in a vacuum oven at 50° C. until a constant weight is achieved, recording its initial dry weight, and then extracting the sample in a boiling solvent such as o-xylene for 24 hours using, e.g., a Soxhlet extractor. After 24 hours, the solvent is removed from the insoluble material, and then the insoluble material is further dried in a vacuum oven at 50° C. until a constant weight is achieved. The gel fraction is determined by dividing the dry weight of the insoluble material by the total initial dry weight of a sample.

In embodiments, the thickness T_{M} is less than about 1500 nm, e.g., less than about 1000 mn, less than about 750 mn, less than about 500 nm, less than about 250 mn, less than about 150 nm, less than about 100 nm or less than about 50 nm. In embodiments, the oxidized region 30 can have a thickness T₁ of less than about 5 e.g., less than about 2 nm or less than about 1 nm. In embodiments, the carbonized region 32 can have a thickness T₂ of less than about 500 nm, e.g., less than about 350 nm, less than about 250 nm, less than about 150 nm or less than about 100 mn, and can occur at a depth from outer surface of less than about 10 nm, e.g., less than about 5 nm or less than about 1 nm. In embodiments, the crosslinked region 34 can have a thickness T₃ of less than about 1500 nm, e.g., less than about 1000 nm, or less than about 500 nm, and can occur at a depth from outer surface 22 of less than about 500 nm, e.g., less than about 350 nm, less than about 250 nm or less than about 100 mm.

In embodiments, thickness T_{M} is about 1% or less, e.g. about 0.5% or less or 0.05% or more, of the thickness T_{B}. In embodiments, the hard polymer region can enhance the mechanical properties the stent. For example, the stent can be enhanced by providing a relatively thick carbonized or crosslinked region. In embodiments, the thickness T_{M} of the hard polymer region can be about 25% or more, e.g. 50 to 90% of the overall thickness T_{B}. In embodiments, the wall has an overall thickness in the unexpanded state of less than 5.0 mm, e.g., less than 3.5 mm, less than 2.5 mm, less than 2.0 mm or less than 1.0 mm.

The base is, e.g., a polymer, a blend, or a layer structure of polymer that provides desirable properties to the stent. Erodible polymers include, e.g., polyanhydrides, polyorthoesters, polylactides, polyglycolides, polysiloxanes, cellulose derivatives and blends or copolymers of any of these. Additional erodible polymers are disclosed in U.S. Published Patent Application No. 2005/0010275, filed Oct. 10, 2003; U.S. Published Patent Application No. 2005/0216074, filed Oct. 5, 2004; and U.S. Pat. No. 6,720,402, the entire disclosure of each of which is hereby incorporated by reference herein.

The base can be formed from multiple layers of materials, some of which can be bio-stable (if desired). In a particular embodiment, the base is formed by coating a bioerodible stent with a polymeric material. The coating material can be made of the same material as the base, or it can be made of a different material. The coating material can be bioerodible or bio-stable. If desired, more than one coating layer can be applied to the bioerodible stent. Such a coating can be applied to the bioerodible stent, e.g., by spray or dip-coating the bioerodible stent. The base and/or coating can also be formed by coextrusion. The base can also be a bioerodible or biostable metal, ceramic or polymer/ceramic composite. Bioerodible metals are discussed in Kaese, Published U.S. Patent Application No. 2003/0221307, Stroganov, U.S. Pat. No. 3,687,135, Heublein, U.S. Published Patent Application No. 2002/0004060; bioerodible ceramics are discussed in Zimmermann, U.S. Pat. No. 6,908,506 and Lee, U.S. Pat. No. 6,953,594; and bioerodible ceramic/polymer composites are discussed in Laurencin, U.S. Pat. No. 5,766,618, the entire disclosure of each of which is hereby incorporated by reference herein. Other non-erodible stent materials include stainless steel and nitinol. The stents described herein can be delivered to a desired site in the body by a number of catheter delivery systems, such as a balloon catheter system. Exemplary catheter systems are described in U.S. Pat. Nos. 5,195,969, 5,270,086, and 6,726,712, the entire disclosure of each of which is hereby incorporated by reference herein. The Radius® and Symbiot® systems, available from Boston Scientific Scimed, Maple Grove, Minn., also exemplify catheter delivery systems. The stent can also be self-expanding.

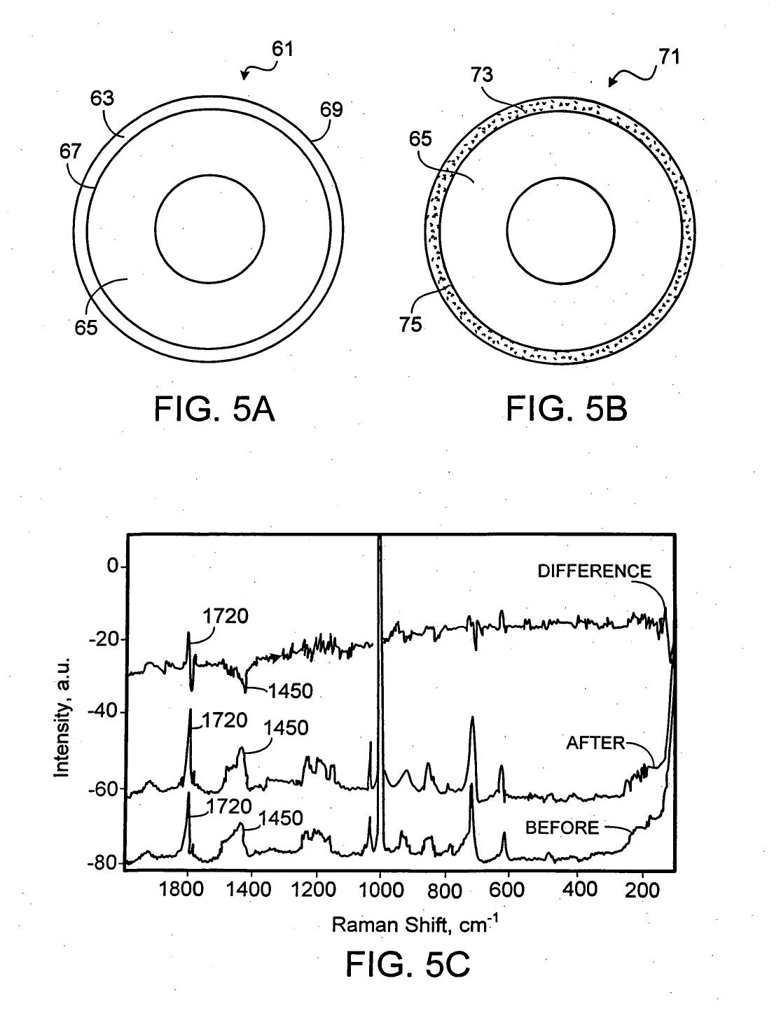

Referring now to FIGS. 5A and 5B, a stent 61 includes a wall 69 that includes a coating layer 63, e.g., formed from a polymer such as a polymer suitable for carrying a therapeutic agent, and a first polymer layer 65 that are bonded at an interface 67. Stent 61 can be modified using PIII to provide a modified stent 71. In the embodiment shown in FIG. 5B, the coating layer 63 and interface 67 of stent 61 is modified with PIII to produce modified layer 73 and modified interface 75 of stent 71. In this particular embodiment, layer 65 is substantially unmodified. Modification of the coating layer 63 of stent 61 provides a hard layer, while modification of the interface 67 enhances adhesion between the adjacent layers in stent 71. Suitable polymers include the bioerodible polymers described above, or non-bioerodible polymers, e.g., PEBAX® and styrenic block copolymers such as styrene-isoprene-butadiene-styrene block copolymer (SIBS). FIG. 5C shows a series of micro-Raman spectra of an outermost surface of a stent having an SIBS coating, the bottom spectrum being before PIII treatment, the middle spectrum being after PIII treatment, and the uppermost spectrum being a difference of the before and after spectra. In this particular embodiment, the stent was treated with N⁺ ions having an energy of 20 keV and a dosage of 10¹⁴ ion/cm². The spectrum after PIII shows a net increase in absorbance in the carbonyl region (centered about 1720 cm⁻¹), and a net decrease in absorbance in the aliphatic region (centered about 1450 cm⁻¹), indicating an increase in oxidation in the outermost surface. A modified SIBS coating can be used to carry and release a therapeutic agent.

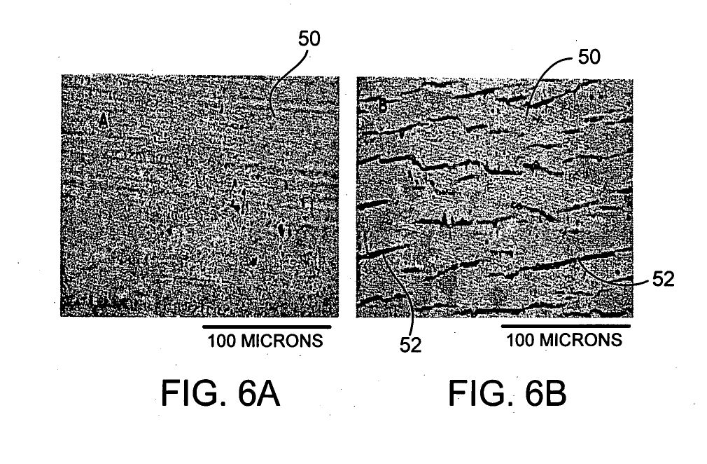

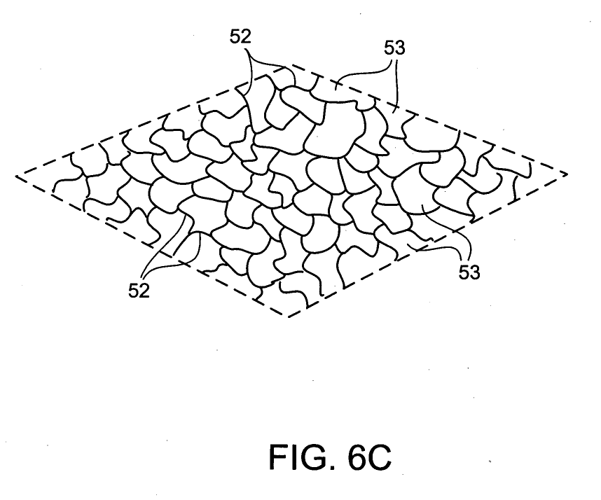

A stent can be modified to provide a desirable surface morphology. Referring to FIG. 6A, a polymeric material surface 50 prior to modification is illustrated to include a relatively flat and featureless polymer profile (polymeric material is formed from PEBAX® 7033). Referring to FIG. 6B, after modification by PIII, the surface includes a plurality of fissures 52. The size and density of the fissures can affect surface roughness, which can enhance the friction between the stent and balloon, improving retention of the stent during delivery into the body. Referring to FIG. 6C, in some embodiments, the fracture density is such that non-fractured “islands” 53 defined by fracture lines 52 are not more than about 20 μm , e.g., not more than about 10 μm , or not more than about 5 μm ². In embodiments, the fracture lines are, e.g., less than 10 μm wide, e.g., less than 5 μm, less than 2.5 μm, less than 1 μm, less than 0.5 μm, or even less than 0.1 μm wide.

The stents can carry a releasable therapeutic agent. For example, the therapeutic agent can be carried within the stent, e.g., dispersed within a bioerodible material from which the stent is formed or dispersed within an outer layer of the stent, such as a coating that forms part of the stent. The therapeutic agent can also be carried on exposed surfaces of the stent. For example, the fissures described above in reference to FIG. 6B can be utilized as a reservoir for a therapeutic agent. In instances in which the fissures are utilized, the therapeutic agent can be applied to the fissures by soaking or dipping.

Therapeutic agents include, e.g., anti-thrombogenic agents, antioxidants, anti-inflammatory agents, anesthetic agents, anti-coagulants and antibiotics. Therapeutic agents can be nonionic, or they can be anionic and/or cationic in nature. The therapeutic agent can be a genetic therapeutic agent, a non-genetic therapeutic agent, or cells. Therapeutic agents can be used singularly, or in combination. An example of a therapeutic agent is one that inhibits restenosis, such as paclitaxel. Additional examples of therapeutic agents are described in U.S. Published Patent Application No. 2005/0216074, the entire disclosure of which is hereby incorporated by reference herein.

When a stent carries a therapeutic agent that is dispersed within a bioerodible material from which the stent is formed or dispersed within an outer layer of the stent, a hard and impermeable modified region as described above can be utilized to help control the manner in which the releasable therapeutic agent is delivered to the body. For example, treating the entire outer surface of such a stent with PIII ensures that the carried therapeutic agent is not delivered directly to the lumen wall in contact with the stent because the drug cannot penetrate through the modified region to get to the lumen wall. In such instances, the therapeutic agent would be delivered only to the fluid that flows through the stent. As another example, treating only portions of the outer surface of such a stent with PIII reduces delivery of the carried therapeutic agent directly to the lumen wall from the treated portions, but is delivered directly to the lumen wall from untreated portions of the stent in contact with the lumen. Such a configuration allows for selective treatment of portions of the lumen wall.

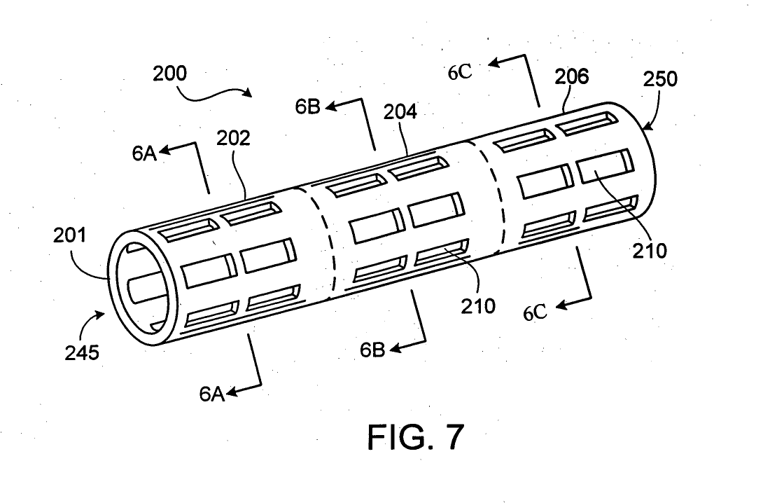

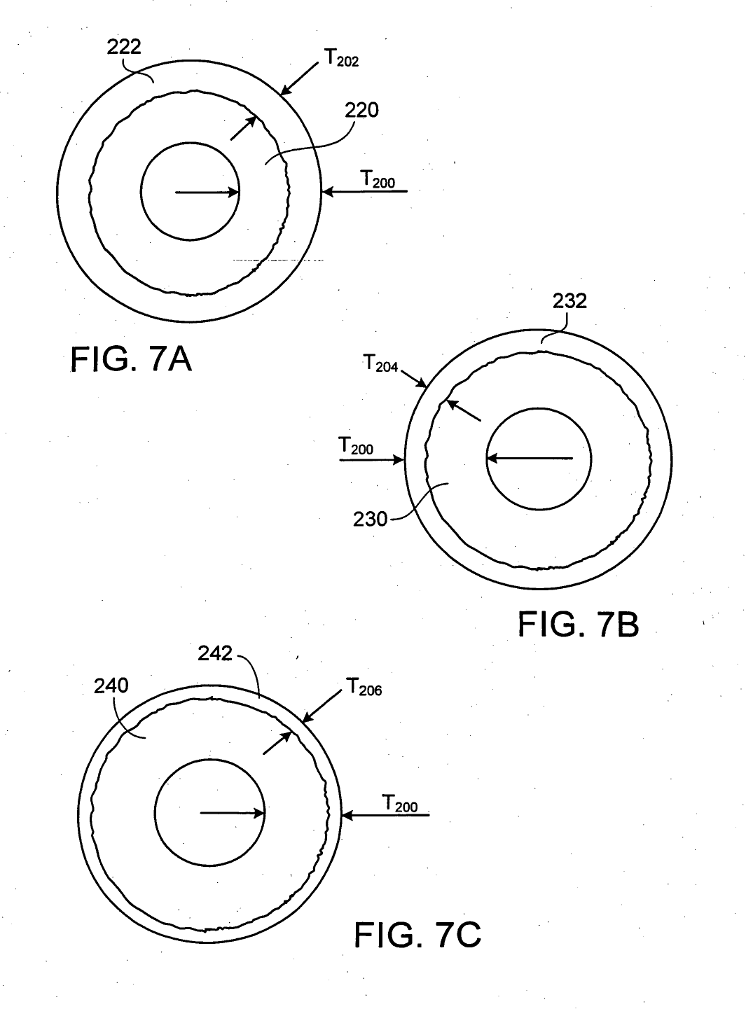

Referring to FIGS. 7-7C, bioerodible stent 200 includes a plurality of fenestrations 210 defined in a wall 201 having a constant thickness T₂₀₀ along its longitudinal length. Stent 200 includes three portions 202, 204 and 206, each portion having a base polymer system and a hard polymer region. In particular, portion 202 has a polymer system 220 and a hard polymer region 222 having thickness T₂₀₂; portion 204 has a polymer system 230 and a hard polymer region 232 having thickness T₂₀₄; and portion 206 has a polymer system 240 and a hard polymer region 242 having thickness T₂₀₆. The thickness of each region becomes smaller when moving from a proximal end 245 to a distal end 250 of the stent (i.e., T₂₀₂>T₂₀₄>T₂₀₆). A configuration such as this allows for control over the manner in which the endoprosthesis erodes, in this case, region 206 completely erodes before region 204, which in turn erodes before region 202. The likelihood of uncontrolled fragmentation is reduced.

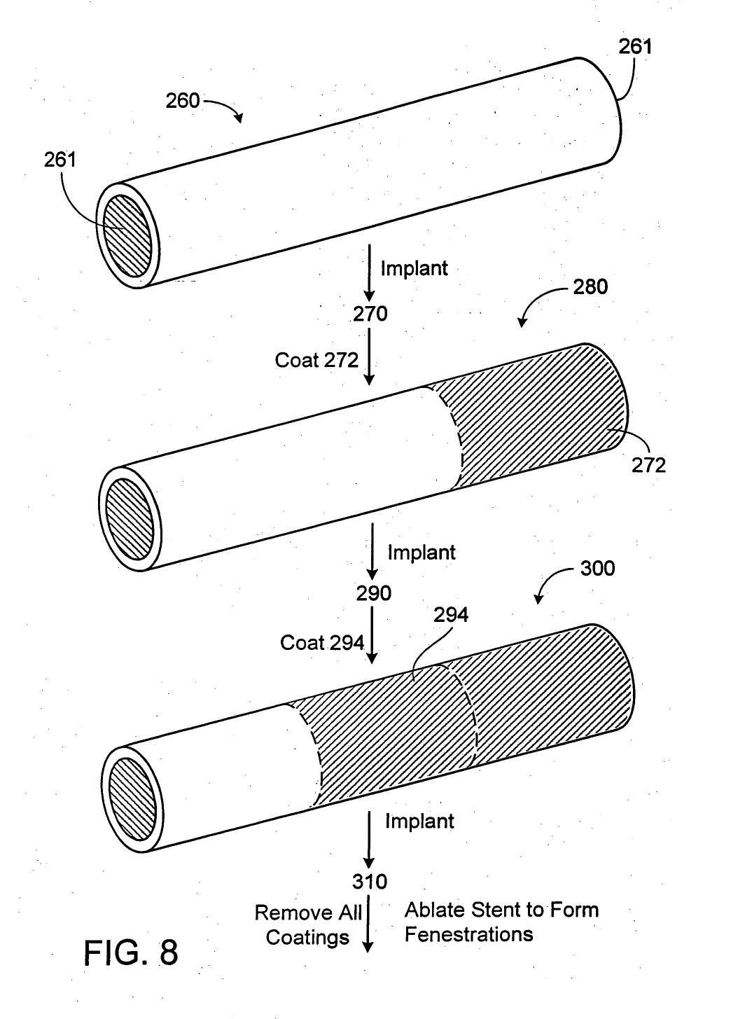

Referring now to FIGS. 7 and 8, bioerodible stent 200 (of FIG. 7) can be produced from an untreated, and un-fenestrated bioerodible pre-stent by employing the PIII system shown in FIGS. 4A-4C. During production, open ends of a tubular pre-stent are plugged with caps 261. Capped pre-stent 260 is placed in the PIII system and all outer portions of the pre-stent are treated with ions. After a desired implantation time, an implanted pre-stent 270 is removed from the PIII system. Implanted pre-stent 270 at this point has a transverse cross-section along its longitudinal length that resembles the cross-section shown in FIG. 7C. Next, all exposed surfaces of portion 272 of implanted pre-stent 270 are covered with a protective polymeric coating, such as PEBAX® or styrene-isoprene-butadiene-styrene (SIBS) copolymer, to produce coated pre-stent 280. Pre-stent 290 is produced by placing pre-stent 280 in the PIII system and ion implanting under conditions such that the ions penetrate more deeply into pre-stent 280 than during formation of pre-stent 270. The coating on portion 272 protects this segment from additional implantation. Next, all exposed surfaces of portion 294 are covered with a coating to produce coated pre-stent 300. Coated pre-stent 300 is then placed back into the PIII system and implanted, producing pre-stent 310. Conditions for implantation are selected such that the ions penetrate more deeply into the uncovered portion than during formation of pre-stent 290. The coating on portions 272 and 294 protect these portions from additional implantation. All coatings are removed, e.g., by rinsing with a solvent such as toluene, fenestrations are cut in the wall of the device, e.g., by laser ablation using an excimer laser operating at 193 nm, and the caps 261 are removed to complete the production of stent 200.

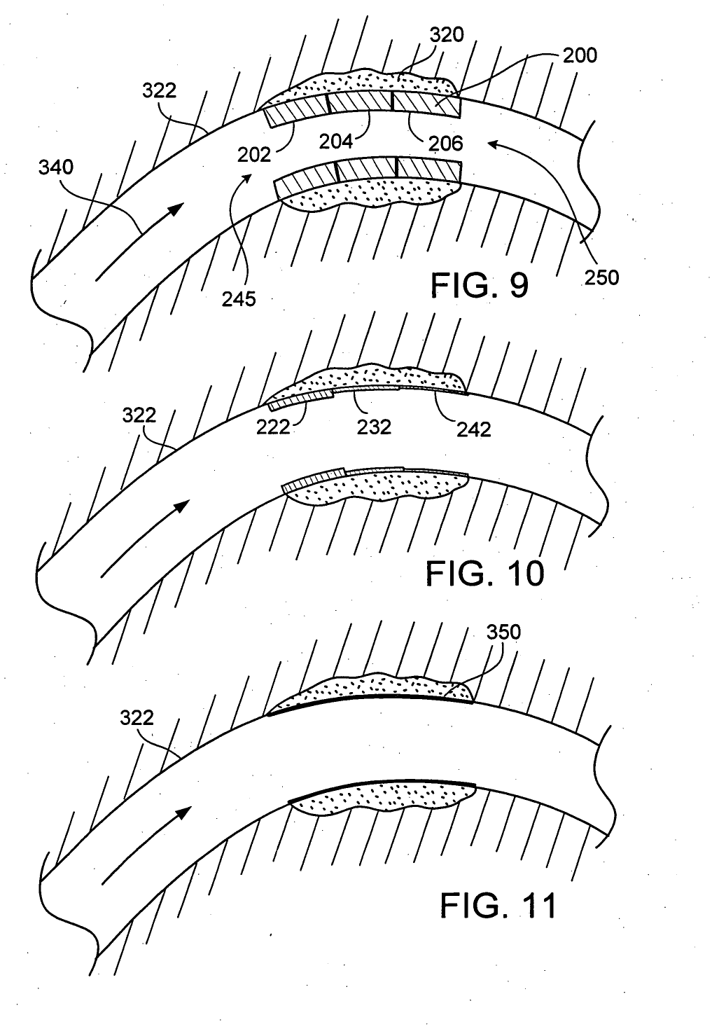

Referring now to FIGS. 7-7C and 9-11, after delivery of the bioerodible stent 200 to the desired site, and expansion and deployment of the stent adjacent occlusion 320, the stent 200 begins to erode within lumen 322. During its deployment, the stent was positioned within the lumen 322 such that end 245 is upstream of end 250 in a flow of fluid in the lumen (direction indicated by arrow 340). The stent erodes from the inside towards the outside because regions 222, 232 and 242 of portions 202, 204 and 206, respectively, prevent intrusion of bodily fluids into the stent from the outside towards the inside. In the early stages of erosion, the hard surfaces provided by the stent support cellular growth (endothelialization) and allow the stent to become firmly anchored within the lumen. After erosion of the base polymer system of each portion 202, 204 and 206, only the regions 222, 232 and 242 of portions 202, 204 and 206, respectively, remain (FIG. 10). At this point, the erosion rate of all the portions slow because the rate of erosion of the crosslinked portion is slower than the base polymer. This allows further cellular growth around the remnants of the stent. In late stages of erosion (FIG. I 1), only the oxidized and carbonized regions (collectively 350) remain, which are fully enveloped with cell growth and anchored to the lumen.

The stents described herein can be configured for vascular or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radio-opaque by addition of, e.g., radio-opaque materials such as barium sulfate, platinum or gold, or by coating with a radio-opaque material.

A number of embodiments have been described. Nevertheless, it will be understood that various modifications may be made without departing from the spirit and scope of the disclosure. Accordingly, other embodiments are within the scope of the following claims.

What is claimed is:

1. An endoprosthesis comprising an endoprosthesis wall having a bioerodible base and a region including carbonized polymer material.

2. The endoprosthesis of claim 1, wherein the base is a bioerodible polymer system.

3. The endoprosthesis of claim 1, wherein the carbonized polymer material is an integral modified region of the base bioerodible polymer system.

4. The endoprosthesis of claim 1, wherein the base is a bioerodible metal.

5. The endoprosthesis of claim 1, wherein the region includes a diamond-like carbon material.

6. The endoprosthesis of claim 1, wherein the region includes a graphitic carbon material.

7. The endoprosthesis of claim 1, wherein the region includes a region of crosslinked base polymer material.

8. The endoprosthesis of claim 7, wherein the crosslinked region is directly bonded to the carbonized polymer material and to substantially unmodified base polymer material.

9. The endoprosthesis of claim 1, including a region of oxidized polymer material, the oxidized region being directly bonded to the carbonized material without further bonding to the base.

10. The endoprosthesis of claim 1, wherein the region extends from a surface of the base.

11. The endoprosthesis of claim 1, wherein an overall modulus of elasticity of the base is within about +/−10% of the base polymer system without the region.

12. The endoprosthesis of claim 1, wherein a thickness of the region is about 10 nm to about 2000 nm.

13. The endoprosthesis of claim 1, wherein the region has a thickness that is about 20% or less than an overall thickness of the base polymer system.

14. The endoprosthesis of claim 1, wherein the base polymer is selected from the group consisting of polyester amides, polyanhydrides, polyorthoesters, polylactides, polyglycolides, polysiloxanes, cellulose derivatives, and copolymers and blends thereof.

15. The endoprosthesis of claim 1, wherein the base is a metal.

16. The endoprosthesis of claim 15, wherein the metal is selected from the group consisting of magnesium, calcium, lithium, rare earth elements, iron, aluminum, zinc, manganese, cobalt, copper, zirconium, titanium, and mixtures thereof.

17. The endoprosthesis of claim 1, wherein the region has a fractured surface morphology.

18. The endoprosthesis of claim 1, wherein the region carries a therapeutic agent.

19. The endoprosthesis of claim 1, wherein the base includes a coating.

20. An endoprosthesis exhibiting a D peak.

21. The endoprosthesis of claim 20, wherein the endoprosthesis also exhibits a G peak.

22. The endoprosthesis of claim 20, wherein the endoprosthesis has a first region exhibiting a D peak and a second region exhibiting a G peak.

23. The endoprosthesis of claim 22, wherein the first region and the second region are at different depths through the endoprosthesis.

24. The endoprosthesis of claim 22, wherein the first region and the second region are at different longitudinal or radial location of the endoprosthesis.

25. The endoprosthesis of claim 20, wherein the endoprosthesis carries a therapeutic agent.

26. The endoprosthesis of claim 22, wherein the therapeutic agent is in and/or on a region of the endoprosthesis exhibiting the D peak.

27. A method of making an endoprosthesis, the method comprising:
providing an endoprosthesis that includes a bioerodible base and a polymer; and
treating the polymer by ion implantation.

28. The method of claim 27, wherein the base is a polymer and wherein the base is treated to provide a modified region.

29. The method of claim 27, wherein the bioerodible base is provided with a polymer layer, and wherein the polymer layer is treated to provide a modified region.

30. The method of claim 27, wherein the bioerodible base is a metal.

31. The method of claim 27, including treating the polymer to provide a carbonized polymer.

32. An endoprosthesis formed by the method of claim 27.

33. A method of making an endoprosthesis, the method comprising:
providing an endoprosthesis having a metal base and having a polymer layer; and
treating the polymer layer by ion implantation.

34. The method of claim 33, including treating the polymer layer to form a carbonized region.

35. The method of claim 33, including treating the polymer layer to provide a fractured surface morphology.

36. The method of claim 35, including providing the fractured surface with a therapeutic agent.

37. The method of claim 33, wherein the metal is not bioerodible.
